(19) United States
(12) Patent Application Publication
(10) Pub. No.: US 2011/0300132 A1
(43) Pub. Date: Dec. 8, 2011

(54) 4-AMINOQUINAZOLINE PRODRUGS

(75) Inventors: Roger Tung, Lexington, MA (US); Adam Morgan, Lexington, MA (US)

(73) Assignee: Concert Pharmaceuticals, Inc., Lexington, MA (US)

(21) Appl. No.: 12/986,135

(22) Filed: Jan. 6, 2011

Related U.S. Application Data
(63) Continuation of application No. PCT/US2009/004022, filed on Jul. 8, 2009.
(60) Provisional application No. 61/079,423, filed on Jul. 9, 2008.

(51) Int. Cl. A61K 39/395; A61K 31/675; A61P 35/02; A61K 33/24; A61P 35/00; C07F 9/09; A61K 38/22

(52) U.S. Cl. 424/133.1; 544/244; 514/80; 514/9.7; 424/649

(57) ABSTRACT

This invention relates to prodrugs of 4-aminoquinazoline compounds, and to pharmaceutically acceptable salts thereof. This invention also provides compositions comprising a compound of this invention and the use of such compositions in methods of treating diseases and conditions that are beneficially treated by administering inhibitors of the EGFR and HER2.

### RELATED APPLICATIONS

This application is a continuation of PCT International Application No. PCT/US09/04022, filed Jul. 8, 2009, which claims the benefit of U.S. Provisional Patent Application No. 61/079,423, filed Jul. 9, 2008. The entire contents of each of these applications are incorporated herein by reference.

This invention relates to prodrugs of both 4-aminoquinazoline compounds and deuterated 4-aminoquinazoline compounds, and to pharmaceutically acceptable salts thereof. This invention also provides compositions comprising a compound of this invention and the use of such compositions in methods of treating diseases and conditions that are beneficially treated by administering inhibitors of the EGFR and HER2.

Lapatinib, also known as N-[3-Chloro-4-(3-fluorobenzyloxy)phenyl]-6-[5-[2-(methylsulfonyl)ethylaminomethyl]furan-2-yl]quinazolin-4-amine bis(4-methylbenzenesulfonate) hydrate, inhibits the tyrosine kinase activity of both the Epidermal Growth Factor Receptor (EGFR; ErbB1) and the human epidermal receptor Type 2 (HER2; ErbB2). Lapatinib has been approved in the United States in combination with capecitabine for the treatment of patients with advanced or metastatic breast cancers whose tumors over-express HER2 and who have failed prior therapy.

Lapatinib is both metabolized by and also inhibits cytochrome P450 subtype 3A4 (CYP 3A4) at clinically relevant concentrations. The FDA approval label suggests avoiding co-dosing with strong CYP3A4 inhibitors or reducing the dose of lapatinib in patients requiring administration of compounds that are CYP3A4 inhibitors (http://www.fda.gov/cder/foi/label/2007/0220591b1.pdf). It is noteworthy that gastrointestinal toxicity, a clinically limiting aspect of the drug, appears to be related to the amount dosed rather than to plasma concentrations (Burris H A et al., J Clin Oncol 2005; 23:5305). This suggests that local drug concentrations in the gut are responsible for lapatinib's toxicity and that increasing plasma concentrations for a given oral dose is likely to increase its therapeutic window and therefore enhance its utility without resulting in an associated increase in adverse side-effects.

Compounds that are chemically related to lapatinib have also been described and have been shown to have potent tyrosine kinase inhibitory activity against ErbB1, ErbB2 and/or ErbB4 (HER4). See U.S. Pat. No. 6,727,256.

Despite the beneficial activities of lapatinib, there is a continuing need for new compounds to treat the aforementioned diseases and conditions.

### DEFINITIONS

The term “treat” means decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of lapatinib will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the naturally occurring abundance of a specified isotope.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

The term “isotopologue” refers to a species that differs from a specific compound of this invention only in the isotopic composition thereof.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound. In other embodiments, the relative amount of such isotopologues in toto will be less that 47.5%, less than 40%, less than 32.5%, less that 25%, less than 17.5%, less than 10%, less than 5%, less than 3%, less than 1%, or less than 0.5% of the compound. The term “compound,” as used herein, is also intended to include any salts thereof.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

Suitable cationic moieties to form pharmaceutically acceptable salts of the phosphonic acid residue of the compounds of this invention include, but are not limited to, alkali metals such as sodium, potassium, and lithium; alkaline earth metals such as calcium and magnesium; other metals, such as aluminum and zinc; ammonia, and organic amines, such as mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; amino acids such as arginine, lysine, and the like, and zwitterions, such as glycine and the like.

In one embodiment, the cationic moiety used to form pharmaceutically acceptable salts of the phosphonic acid residue of the compounds of this invention is selected from alkali metals and alkaline earth metals. In a more specific embodiment, the cationic moiety is selected from sodium, magnesium and calcium. In a further specific embodiment, the cationic moiety is sodium.

The disclosed compounds may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are minor images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms.

When the stereochemistry of the disclosed compounds is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% optically pure. Percent optical purity by weight is the ratio of the weight of the enantiomer over the weight of the enantiomer plus the weight of its optical isomer.

When a disclosed compound is named or depicted by structure without indicating the stereochemistry, and has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer (“scalemic mixtures”).

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X” % of other stereoisomers (wherein X is a number between 0 and 100, inclusive) are present.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert” and “t-” each refer to tertiary. “US” refers to the United States of America. “FDA” refers to Food and Drug Administration. “NDA” refers to New Drug Application.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

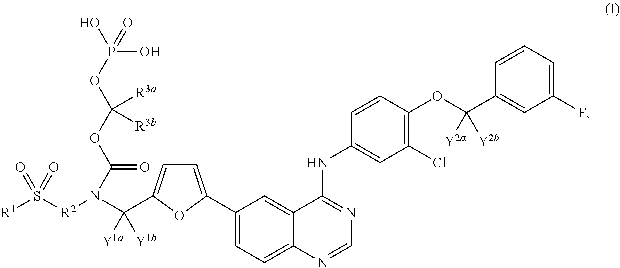

or a
pharmaceutically acceptable salt thereof, wherein:

R¹ is selected from —CH₃, —CH₂D, —CHD₂, and —CD₃;

R² is an ethylene moiety having 0 to 4 deuterium atoms;

each of R^{3a} and R^{3b} is independently selected from hydrogen, C₁-C₆ straight or branched alkyl and C₃-C₇ cycloalkyl, or

R^{3a} and R^{3b} are taken together with the carbon atom to which they are bound to form a C₃-C₇ cycloalkyl,

wherein any alkyl or cycloalkyl group in R^{3a} or R^{3b} or formed by R^{3a} and R^{3b} taken together is optionally substituted with halo, C₁₋₇ alkyl, cyano, hydroxyl, carboxy, alkoxy, oxo, amino, alkylamino, or dialkylamino; and

each Y is independently selected from hydrogen and deuterium.

One embodiment of Formula I provides a compound wherein R¹ or R² has at least one deuterium or at least one Y is deuterium.

In one embodiment R¹ is —CH₃ or —CD₃.

In one embodiment, the moiety in Formula I represented by

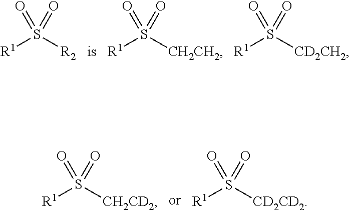

In one embodiment, R^{3a} and R^{3b} are both hydrogen or C₁-C₆ straight or branched alkyl, or are taken together to form a C₃-C₇ cycloalkyl.

In one embodiment, R^{3a} and R^{3b} are hydrogen.

In one embodiment, Y^{1a} and Y^{1b} are the same; and Y^{2a} and Y^{2b} are the same.

In one embodiment, R¹ is —CH₃ or —CD₃; and R^{3a} and R^{3b} are both hydrogen. In one aspect of this embodiment where R¹ is —CH₃ or —CD₃ and R^{3a} and R^{3b} are both hydrogen, Y^{1a} and Y^{1b} are both hydrogen and Y^{2a} and Y^{2b} are both hydrogen. In another aspect of this embodiment, Y^{1a} and Y^{1b} are both hydrogen, and Y^{2a} and Y^{2b} are both deuterium. In another aspect of this embodiment, Y^{1a} and Y^{1b} are both deuterium, and Y^{2a} and Y^{2b} are both hydrogen. In another aspect of this embodiment, Y^{1a} and Y^{1b} are both deuterium, and Y^{2a} and Y^{2b} are both deuterium.

In one embodiment, pharmaceutically acceptable salts of a compound of Formula I include a disodium, dipotassium, diammonium, calcium, or magnesium salt. In another embodiment, the invention provides a compound of Formula II:

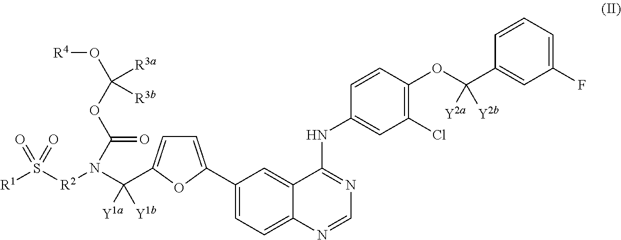

wherein R¹, R², R^{3a}, R^{3b}, Y^{1a}, Y^{1b}, Y^{2a}, and Y^{2b} are as defined above; and

R⁴ is —PO₃H₂, —PO₃Na₂, —PO₃K₂, —PO₃(NH₄)₂, —PO₃Ca, or —PO₃Mg.

In one embodiment, the compound is selected from any one of the compounds set forth in Table 1, wherein R¹ is CH₃ and R^{3a} and R^{3b} are both hydrogen.

TABLE 1
Examples of Specific Compounds of Formula II

| Compound | R²-†, | Y^{1a}═Y^{1b} | Y^{2a}═Y^{2b} | R⁴ |
|---|---|---|---|---|
| 101 | CH₂CH₂—† | H | H | —PO₃H₂ |
| 102 | CH₂CH₂—† | H | D | —PO₃H₂ |
| 103 | CH₂CH₂—† | H | H | —PO₃Na₂ |
| 104 | CH₂CH₂—† | H | D | —PO₃Na₂ |
| 105 | CH₂CH₂—† | D | H | —PO₃H₂ |
| 106 | CH₂CH₂—† | D | D | —PO₃H₂ |
| 107 | CH₂CH₂—† | D | H | —PO₃Na₂ |
| 108 | CH₂CH₂—† | D | D | —PO₃Na₂ |
| 109 | CD₂CH₂—† | H | H | —PO₃H₂ |
| 110 | CD₂CH₂—† | H | D | —PO₃H₂ |
| 111 | CD₂CH₂—† | H | H | —PO₃Na₂ |
| 112 | CD₂CH₂—† | H | D | —PO₃Na₂ |
| 113 | CD₂CH₂—† | D | H | —PO₃H₂ |
| 114 | CD₂CH₂—† | D | D | —PO₃H₂ |
| 115 | CD₂CH₂—† | D | H | —PO₃Na₂ |
| 116 | CD₂CH₂—† | D | D | —PO₃Na₂ |
| 117 | CH₂CD₂-† | H | H | —PO₃H₂ |
| 118 | CH₂CD₂-† | H | D | —PO₃H₂ |
| 119 | CH₂CD₂-† | H | H | —PO₃Na₂ |
| 120 | CH₂CD₂-† | H | D | —PO₃Na₂ |
| 121 | CH₂CD₂-† | D | H | —PO₃H₂ |
| 122 | CH₂CD₂-† | D | D | —PO₃H₂ |
| 123 | CH₂CD₂-† | D | H | —PO₃Na₂ |
| 124 | CH₂CD₂-† | D | D | —PO₃Na₂ |
| 125 | CD₂CD₂-† | H | H | —PO₃H₂ |
| 126 | CD₂CD₂-† | H | D | —PO₃H₂ |
| 127 | CD₂CD₂-† | H | H | —PO₃Na₂ |
| 128 | CD₂CD₂-† | H | D | —PO₃Na₂ |
| 129 | CD₂CD₂-† | D | H | —PO₃H₂ |
| 130 | CD₂CD₂-† | D | D | —PO₃H₂ |
| 131 | CD₂CD₂-† | D | H | —PO₃Na₂ |
| 132 | CD₂CD₂-† | D | D | —PO₃Na₂ |

“†” indicates point of attachment to the nitrogen atom

Examples of specific compounds of Formula I and II include the following:

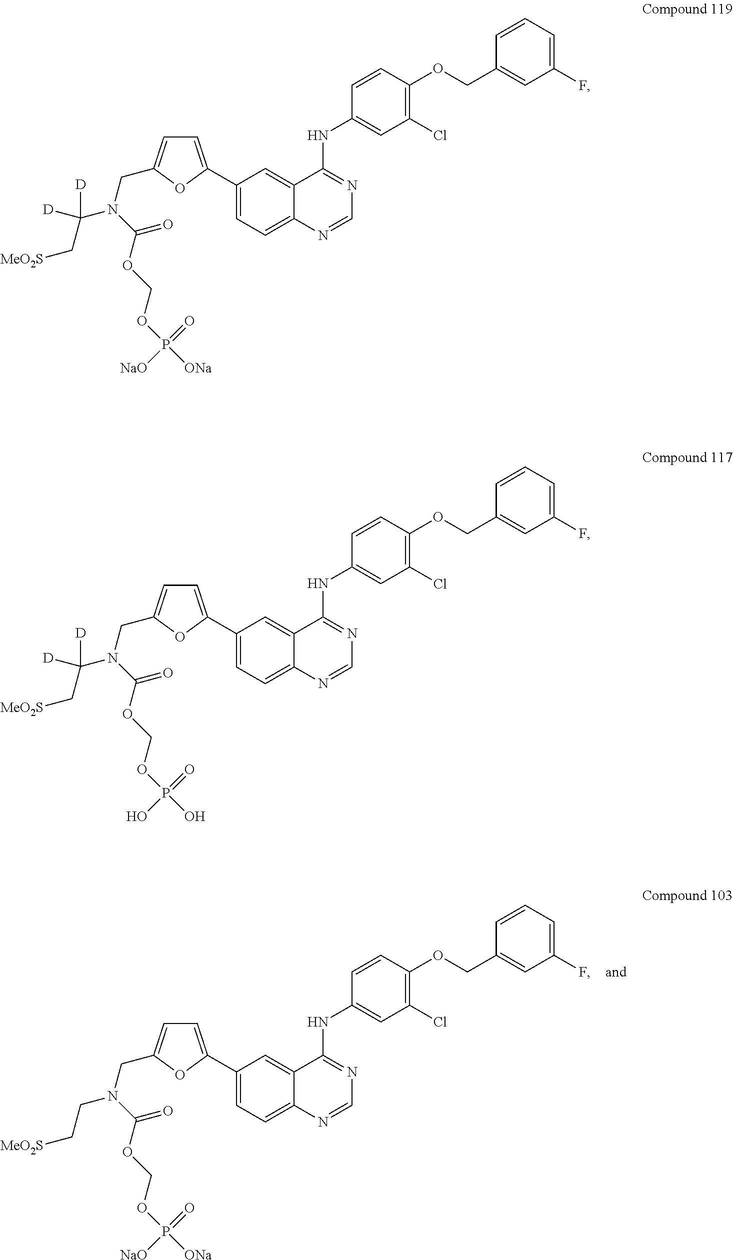

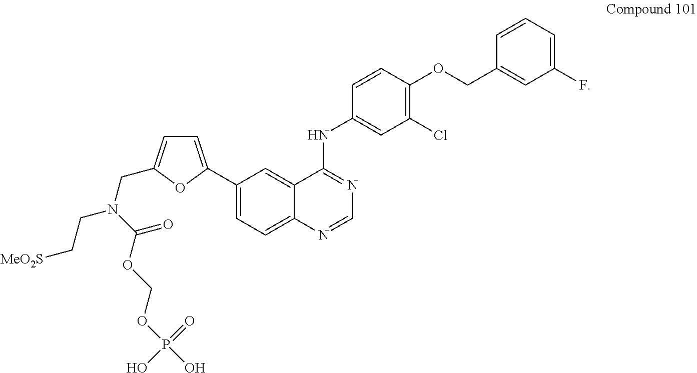

In another set of embodiments, any atom not designated as deuterium in any of the embodiments set forth above is present at its natural isotopic abundance.

The synthesis of compounds of Formula I can be readily achieved by synthetic chemists of ordinary skill with reference to the Schemes and Examples disclosed herein.

### Exemplary Synthesis

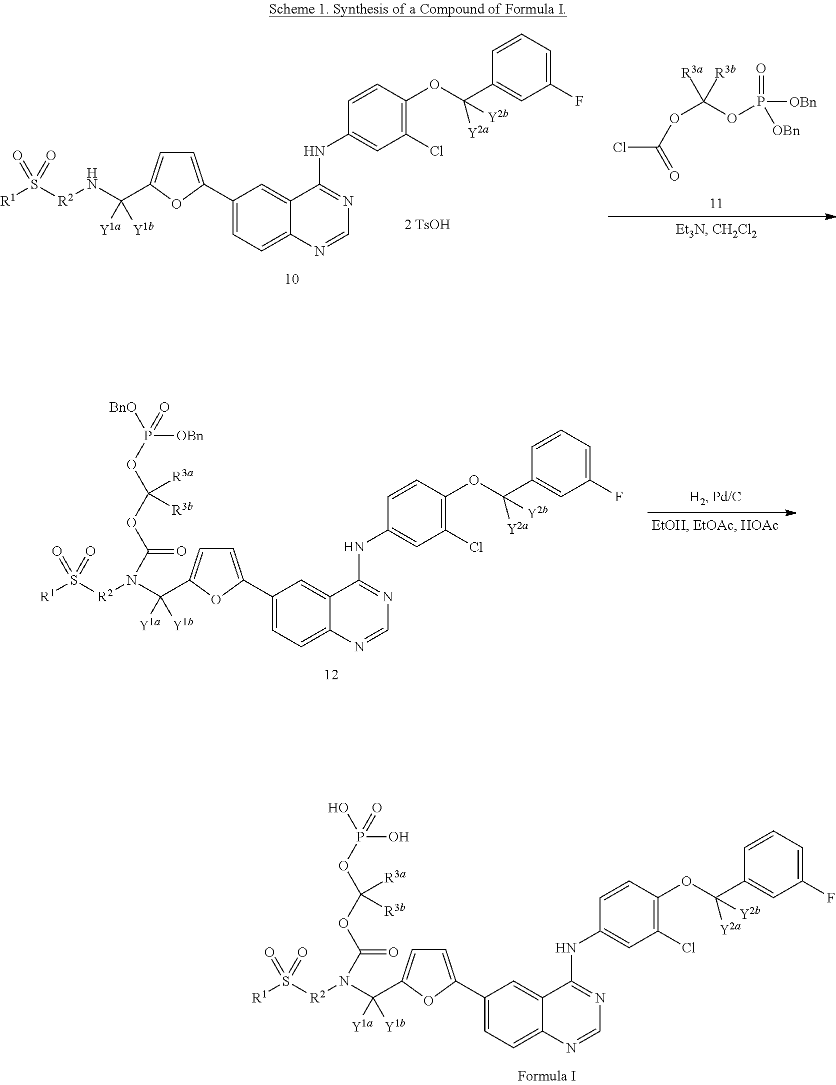

A convenient method for synthesizing compounds of Formula I is depicted in Scheme 1. Deuterated or undeuterated lapatinib ditosylate 10 may be combined with a bis(benzyloxy)phosphoryloxy)carbonochloridate 11 (prepared as disclosed in European patent publication 0 747 385) in dichloromethane and triethylamine to form benzyl-protected intermediate 12. Intermediate 12 may be deprotected by reduction over Pd/C to produce the phosphonic acid of Formula I. Salts of the phosphonic acid of Formula I may be formed by treatment with the appropriate hydroxide in an organic solvent or by treatment of the phosphonic acid with an appropriate Chelex® cation exchange resin.

The synthesis of appropriately deuterated lapatinib ditosylates 10 are described in United States patent publication US 2008/0051422. The synthesis of an undeuterated lapatinib ditosylate 10 is described in U.S. Pat. No. 6,727,256.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Combinations of substituents and variables envisioned by this invention are only those that result in the formation of stable compounds.

### Compositions

The invention also provides pyrogen-free compositions comprising an effective amount of a compound of Formula I or II, or a pharmaceutically acceptable salt of said compound and an acceptable carrier.

Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

In a more specific embodiment, the composition suitable for oral administration will comprise an enteric coat around the compound of Formula I. An enteric coat is a coat made of a material that prevents the compound from being released in the lower pH areas of the gastrointestinal tract, but allows release as the dosage form moves to the higher pH areas of the gastrointestinal tract. For certain compounds of this invention exposure to the higher pH areas of the gastrointestinal tract will increase solubility. Moreover, prevention of exposure of the compound to the lower pH areas of the gastrointestinal tract may be beneficial in reducing gastric side effects known to exist for lapatinib.

Enteric coats may be applied by a number of traditional methods including, but no limited to, conventional coating procedures or fluid bed spraying. Suitable enteric coating materials include one or more polymers.

Examples include methacrylic acid copolymers, cellulose acetate butyrate, cellulose acetate trimellitate, carboxymethylethylcellulose, shellac, Eudragit L, Eudragit S (Rohm Pharma), hydroxypropyl methylcellulose acetate succinate, hydroxypropyl methylcellulose phthalate, polyvinyl acetate phthalate, cellulose acetate phthalate, and the like. Eudragit L and Eudragit S are tradenames of co-polymerized methacrylic acid/methacrylic acid methyl esters.

The enteric coat may contain pharmaceutically acceptable plasticizers to obtain desirable mechanical properties, such as flexibility and hardness of the enteric coating layers. Such plasticizers are for instance, but not restricted to, triacetin, citric acid esters, triethyl citrate, phthalic acid esters, diethyl phthalate, dibutyl sebacate, cetyl alcohol, polyethylene glycols, polysorbates or other plasticizers. Additives such as dispersants, colorants, pigments, polymers e.g. poly(ethylacrylate, methylmethacrylate), anti-tacking and anti-foaming agents may also be included in the enteric coat. Other compounds may be added to increase film thickness and to decrease diffusion of acidic gastric juices into the acidic susceptible active substance.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluoro silicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as Lapatinib. Such agents include, but are not limited to, additional anti-neoplastic agents such as anti-microtubule agents, such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclines, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

In one embodiment, the second therapeutic agent is selected from capecitabine, pazopanib, trastuzumab, docetaxel, letrozole, tamoxifen, fulvestrant, paclitaxel, carboplatin, bevacizumab, doxorubicin, cyclophosphamide, cisplatin, vinorelbine, everolimus, valproic acid, topotecan, oxaliplatin and gemcitabine.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from for example, 0.5 mg to 1200 mg, preferably 1 mg to 1000 mg, more preferably 5 mg to 400 mg of a compound of Formula I or II, per unit dose form depending on the condition being treated, the route of administration and the age, weight and condition of the patient, or pharmaceutical formulations may be presented in unit dose forms containing a predetermined amount of active ingredient per unit dose. In an alternate embodiment, a unit dosage formulation of a compound of this invention may contain between about 100 mg and 2,000 mg of a compound of Formula I or II; or between about 250 mg and 1500 mg of a compound of Formula I or II. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for Lapatinib.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In one embodiment, the invention provides a method of inhibiting ErbB-1, ErbB-2, or ErbB-4-associated protein kinase activity in a cell comprising the step of contacting the cell with a compound of Formula I or II, or a pharmaceutically acceptable salt thereof.

In another embodiment, the present invention provides methods of treating a subject suffering from or susceptible to a neoplastic disease. While compounds of Formula I or II or a pharmaceutically acceptable salt thereof are particularly useful for the treatment of breast cancer, particularly metastatic breast cancer, the invention is not so limited. Illustrative neoplasms for which the invention can be used include, but are not limited to, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

In a specific embodiment, the subject is suffering from or susceptible to a breast cancer, esophageal adenocarcinoma, esophageal squamous cell carcinoma, cervical cancer, head and neck cancer, solid tumors, non-Hodgkins' Lymphoma, gastric cancer, ovarian cancer, peritoneal cancer, brain and CNS tumors (glioma, glioblastoma multiforme, gliosarcoma), prostate cancer, endometrial cancer, colorectal cancer, non-small cell lung cancer, liver cancer, renal cancer, pancreatic cancer,

In another aspect of the present invention, there is provided a method of treating an ErbB2, ErbB4, or EGF (ErbB1) receptor positive neoplasia in a mammal. In a specific embodiment, the subject is suffering from or susceptible to an ErbB positive breast cancer. In a more specific embodiment, the breast cancer is ErbB2, ErbB4, or EGF receptor positive or overexpressing. In an even more specific embodiment, the breast cancer is ErbB2, or EGF receptor positive. In another more specific embodiment, the breast cancer is not responsive to convention chemotherapies, and/or disorders or symptoms thereof. These methods comprise the step of administering to the patient a therapeutically effective amount of a pharmaceutical composition comprising a compound of Formula I or II or a pharmaceutically acceptable salt thereof to a subject (e.g., a mammal such as a human) in need thereof. A “therapeutically effective amount” of a compound herein is an amount sufficient to treat the disease or disorder or symptom thereof.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents or other therapeutic treatments. The choice of second therapeutic agent or therapeutic treatment may be made from any second therapeutic agent or treatment known to be useful for co-administration with lapatinib. The choice of second therapeutic agent or treatment is also dependent upon the particular disease or condition to be treated. Examples of second therapeutic agents that may be employed in the methods of this invention are those set forth above for use in combination compositions comprising a compound of this invention and a second therapeutic agent. Examples of therapeutic treatments include radiation treatments.

In one embodiment, the method of treating a subject suffering from or susceptible to a cancer comprises the additional step of administering to the subject in need thereof a second therapy selected from an anti-neoplastic therapy (which may be selected from a second therapeutic agent other than a compound of Formula I or II, and a therapeutic treatment) and an immunosuppressant.

In one specific embodiment, the subject is suffering from or susceptible to breast cancer and the second therapy is selected from capecitabine, pazopanib, trastuzumab, docetaxel, letrozole, tamoxifen, fulvestrant, paclitaxel, carboplatin, bevacizumab, doxorubicin and cyclophosphamide.

In another specific embodiment, the subject is suffering from or susceptible to cervical cancer and the second therapy is pazopanib.

In still another specific embodiment, the subject is suffering from or susceptible to head and neck cancer and the second therapy is selected from radiation treatment and cisplatin.

In another specific embodiment, the subject is suffering from or susceptible to solid tumors and the second therapy is selected from vinorelbine, everolimus, paclitaxel, valproic acid, docetaxel and topotecan.

In another specific embodiment, the subject is suffering from or susceptible to non-Hodgkin's lymphoma and the second therapy is everolimus.

In another specific embodiment, the subject is suffering from or susceptible to gastric cancer and the second therapy is paclitaxel.

In another specific embodiment, the subject is suffering from or susceptible to ovarian cancer and the second therapy is selected from carboplatin and topotecan.

In another specific embodiment, the subject is suffering from or susceptible to malignant glioma and the second therapy is pazopanib.

In another specific embodiment, the subject is suffering from or susceptible to peritoneal cancer and the second therapy is topotecan.

In another specific embodiment, the subject is suffering from or susceptible to pancreatic cancer and the second therapy is selected from oxaliplatin and gemcitabine.

The term “co-administered” as used herein in connection with a second therapeutic agent means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

The term “co-administered” as used herein in connection with a treatment means that the treatment may be administered to the patient prior to, during, or following any administration to the patient of a composition comprising a compound of Formula I or II, or a pharmaceutically acceptable salt thereof.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I or II, or a pharmaceutically acceptable salt thereof, alone or together with one or more of the above-described second therapeutic agents in the manufacture of a medicament, either as a single composition or as separate dosage forms, for treatment or prevention in a patient of a disease, disorder or symptom set forth above. In still another aspect, the invention provides the use of a compound of Formula I or II, or a pharmaceutically acceptable salt thereof, alone or together with one or more of the above-described second therapeutic agents in the manufacture of a medicament, either as a single composition or as separate dosage forms, for treatment or prevention in a patient of a disease, disorder or symptom set forth above.

Another aspect of the invention is a compound of Formula I or II, or a pharmaceutically acceptable salt thereof, for use in the treatment or prevention in a patient of a disease, disorder or symptom thereof delineated herein.

### Pharmaceutical Kits

The present invention also provides kits for use to treat neoplasias (cancer). These kits comprise (a) a pharmaceutical composition comprising a compound of Formula I or II, or a pharmaceutically acceptable salt thereof, wherein said pharmaceutical composition is in a container; and (b) instructions describing a method of using the pharmaceutical composition to treat a neoplasia (cancer). In a specific embodiment, the kit is for use to treat HER-2 positive breast cancer.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In an embodiment of the kits of this invention, the composition comprising the second active agent may be in a vessel or container that is separate from the vessel containing the composition comprising a compound of Formula I or II.

### EXAMPLES

### Example 1a

### Synthesis of Sodium (((5-(4-(3-Chloro-4-(3-fluorobenzyloxy)phenylamino)quinazolin-6-yl)furan-2-yl)methyl)(2-(methylsulfonyl)ethyl)carbamoyloxy)methyl phosphate (103)

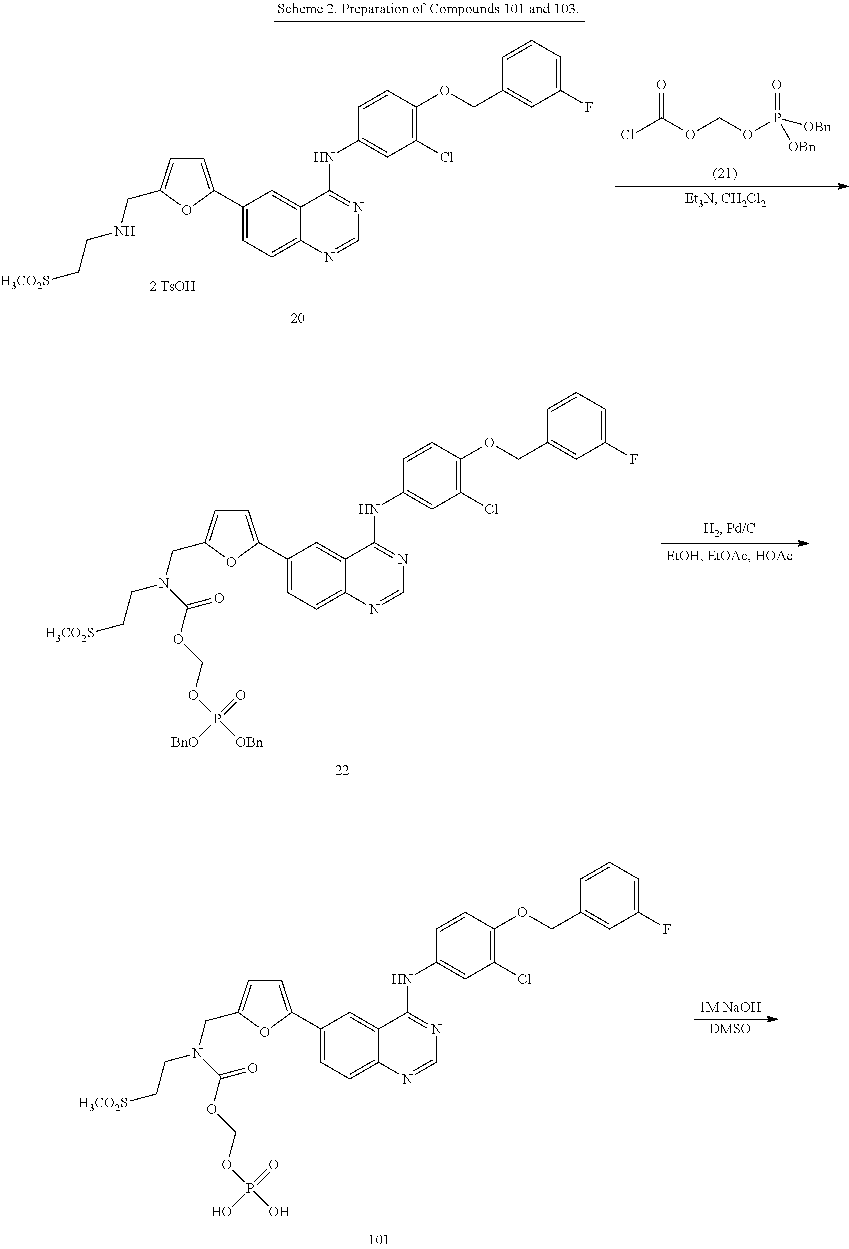

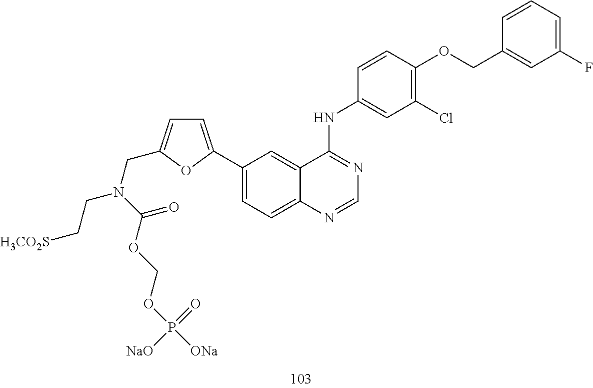

Step 1. (Bis(benzyloxy)phosphoryloxy)methyl (5-(4-(3-chloro-4-(3-fluorobenzyloxy)phenylamino)quinazolin-6-yl)furan-2-yl)methyl(2-(methylsulfonyl)ethyl)carbamate (22). To a suspension of 20 (988 mg, 1.07 mmol, prepared as described in U.S. Pat. No. 6,727,256) in dichloromethane (15.0 mL) was added triethylamine (602 μL, 4.32 mmol). The reaction mixture was stirred at room temperature until a yellow homogeneous solution was obtained (approximately 10 minutes). (Bis(benzyloxy)phosphoryloxy)methyl carbonochloridate (21) (788 mg, 1.62 mmol, prepared according to the procedure of Scola, P M et al., EP 0747385A1) was added and the mixture was stirred for 18 hours. The crude mixture was concentrated in vacuo and the residue purified by column chromatography (silica gel, 0-5% methanol/dichloromethane) to afford pure 22 as a yellow solid (617 mg, 63%). MS (M+H): 915.2.

Step 2. Phosphonooxymethyl (5-(4-(3-chloro-4-(3-fluorobenzyloxy)phenylamino)quinazolin-6-yl)furan-2-yl)methyl(2-(methylsulfonyl)ethyl)carbamate (101). To a solution of 22 (600 mg, 0.656 mmol) in acetic acid (24.0 mL), purged several times with N₂, was added 10% Pd/C (120 mg, 20 wt %). The reaction vessel was evacuated several times with N₂, then was purged several times with H₂ before being placed under an H₂ atmosphere (2 balloons). The reaction mixture was allowed to stir under an atmosphere of H₂ for 15 hours then was purged with N₂. An additional portion of 10% Pd/C (120 mg, 20 wt %) was added and the mixture was again stirred under an H₂ atmosphere for 24 hours. The reaction vessel was then purged with N₂, the crude mixture filtered through Celite®, washed with acetic acid and concentrated in vacuo to afford 101 as a yellow solid (290 mg, 60%). ¹H NMR (400 MHz, DMSO-d₆, apparent rotamers): δ 8.80 (s, 0.5H), 8.75 (s, 0.5H), 8.55 (s, 1H), 8.14 (apparent d, 1H), 8.02 (apparent d, 1H), 7.82-7.70 (m, 2H), 7.47 (apparent q, 1H), 7.38-7.24 (m, 3H), 7.18 (apparent dt, 1H), 7.07 (s, 1H), 6.60 (apparent d, 1H), 5.50 (apparent dd, 2H), 5.26 (s, 2H), 4.64 (s, 2H), 3.76-3.66 (m, 2H) 3.50 (apparent t, 1H), 3.42 (apparent t, 1H), 3.02 (s, 3H). MS (M+H): 735.1.

Step 3. Sodium 4(5-(4-(3-Chloro-4-(3-fluorobenzyloxy)phenylamino)quinazolin-6-yl)furan-2-yl)methyl)(2-(methylsulfonyl)ethyl)carbamoyloxymethyl phosphate (103). To a solution of 101 (68.0 mg, 0.093 mmol) in dimethyl sulfoxide (1.00 mL) was added 1M NaOH (185 μL, 0.185 mmol). After stiffing at room temperature for 1 hour the mixture was frozen and concentrated via lyophilization. The resulting yellow solid was dissolved in H₂O (1.00 mL), frozen, and again concentrated via lyophilization to afford 103 as a yellow solid (62.7 mg, 87%). MS (M+H): 735.1.

### Example 1b

### Alternate Synthesis of (103)

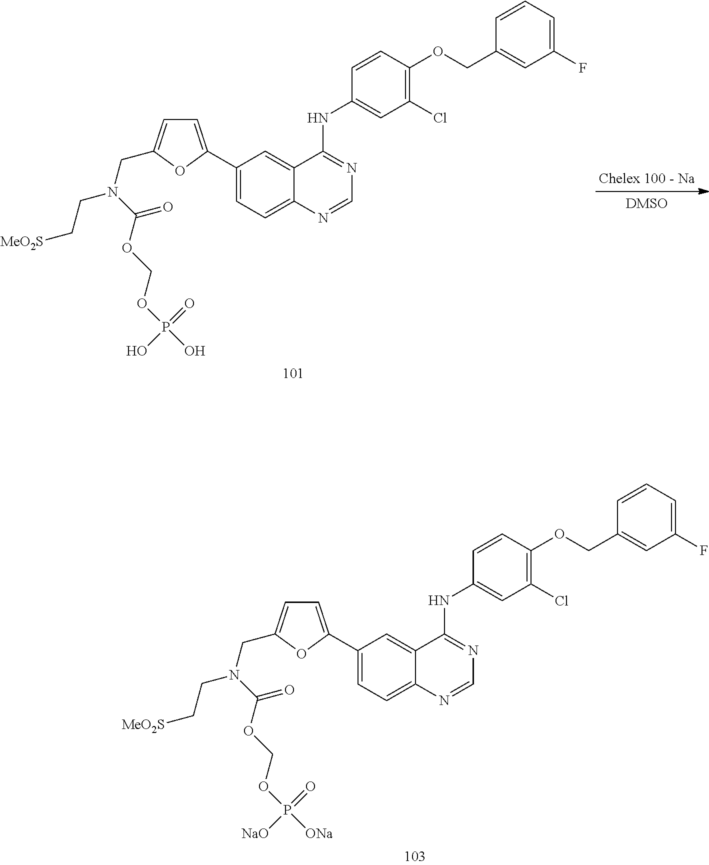

To a solution of Compound 101 (550 mg, 0.748 mmol) in dimethyl sulfoxide (8.00 mL) was added Chelex® 100 sodium form resin (1.50 g, washed with water (3×), methanol (3×), and DMSO (3×)). After stiffing at room temperature for 1 hour the resin was removed by filtration and the process was repeated for a total of 3 treatments. Upon filtration following the third treatment, DMSO was removed via lyophilization. The resulting residue was repeatedly taken up in water (10 mL), frozen, and concentrated via lyophilization until Compound 103 (316 mg, 54%) was obtained as a yellow fluffy solid free of DMSO (lyophilization repeated 3 times). MS (M+H): 735.1.

### Example 2

### Synthesis of Phosphonooxymethyl (5-(4-(3-chloro-4-(3-fluorobenzyloxy)phenylamino)quinazolin-6-yl)furan-2-yl)methyl(2-(methylsulfonyl)-1,1-d₂-ethyl)carbamate (117). Compound 117 was prepared as outlined in Schemes 1 and 2 using appropriately deuterated starting materials

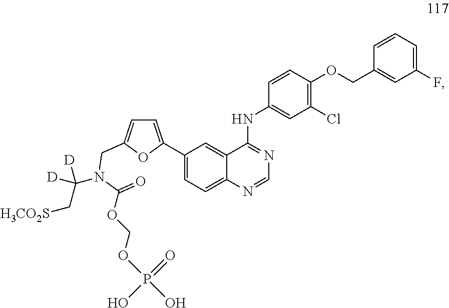

Step 1. (Bis(benzyloxy)phosphoryloxy)methyl (5-(4-(3-chloro-4-(3-fluorobenzyloxy)phenylamino)quinazolin-6-yl)furan-2-yl)methyl(2-(methylsulfonyl)-1,1-d₂-ethyl)carbamate (22-d2). To a suspension of N-(3-chloro-4-(3-fluorobenzyloxy)phenyl)-6-(5-((2-(methylsulfonyl)-1,1-d₂-ethylamino)methyl)furan-2-yl)quinazolin-4-amine (20-d₂) (200 mg, 0.216 mmol, prepared as described in United States patent publication US 2008/0051422) in dichloromethane (3.00 mL) was added triethylamine (120 μL, 0.864 mmol). The reaction mixture was stirred at room temperature until a yellow homogeneous solution was obtained (approximately 10 minutes). (Bis(benzyloxy)phosphoryloxy)methyl carbonochloridate (750 μL, 0.432M in benzene, prepared as described by Scola, P M et al., see Example 1, step 1) was added and the mixture was stirred for 18 hours. The crude mixture was concentrated in vacuo and purified by column chromatography (silica gel, 0-5% methanol/dichloromethane) to afford pure 22-d2 as a yellow solid (90.0 mg, 45%). ¹H NMR (400 MHz, CDCl₃, apparent rotamers): δ 9.10 (s, 0.6H), 8.69 (s, 0.4H), 8.67 (s, 0.6H), 8.55 (s, 0.4H), 8.53 (s, 0.6H), 8.40 (s, 0.4H), 7.96-7.82 (m, 3H), 7.71-7.63 (m, 1H), 7.40-7.12 (m, 12H), 7.05-6.99 (m, 1H), 6.97 (d, J=9.1 Hz, 0.4H), 6.90 (d, J=8.8 Hz, 0.6H), 6.72 (d, J=3.4 Hz, 0.4H), 6.69 (d, J=3.4 Hz, 0.6H), 6.48 (d, J=3.4 Hz, 0.4H), 6.44 (d, J=3.4 Hz, 0.6H), 5.70 (d, J=15.5 Hz, 1.2H), 5.65 (d, J=14.9 Hz, 0.8H), 5.15 (s, 0.8H), 5.09 (s, 1.2H), 5.03-4.90 (m, 4H), 4.60 (s, 0.8H), 4.49 (s, 1.2H), 3.36 (s, 0.8H), 3.26 (s, 1.2H), 2.89 (s, 1.8H), 2.80 (s, 1.2H). MS (M+H): 917.3.

Step 2. Phosphonooxymethyl (5-(4-(3-chloro-4-(3-fluorobenzyloxy)phenylamino)quinazolin-6-yl)furan-2-yl)methyl(2-(methylsulfonyl)-1,1-d₂-ethyl)carbamate (117). To a solution of 22-d2 (134 mg, 0.146 mmol) in acetic acid (5.00 mL), purged several times with N₂, was added 10% Pd/C (27.0 mg, 20 wt %). The reaction vessel was evacuated several times with N₂ then was purged several times with H₂ before being placed under an H₂ atmosphere (2 balloons). The reaction was allowed to stir under an H₂ atmosphere for 15 hours then subsequently purged with N₂. An additional portion of 10% Pd/C (27.0 mg, 20 wt %) was added and the mixture was again stirred under an H₂ atmosphere for 24 hours. The mixture was then purged with N₂, filtered through Celite®, and the resulting acetic acid solution was concentrated in vacuo to afford 117 as a yellow solid (53.0 mg, 49%). ¹H NMR (400 MHz, DMSO-d₆, apparent rotamers): δ 8.88 (s, 0.5H), 8.75 (s, 0.5H), 8.54 (apparent d, 1H), 8.14 (apparent d, 1H), 8.06 (apparent d, 1H), 7.82-7.71 (m, 2H), 7.47 (apparent q, 1H), 7.36-7.14 (m, 4H), 7.07 (s, 1H), 6.60 (apparent d, 1H), 5.50 (apparent dd, 2H), 5.26 (s, 2H), 4.64 (apparent d, 2H), 3.52 (s, 1H), 3.38 (s, 1H), 3.02 (s, 3H). MS (M+H): 737.1.

### Example 3

### Evaluation of Solubility

Aqueous solubility of the compounds of this invention was determined by suspending sufficient compound in different aqueous buffers to give a maximum final concentration of ≧10 mg/mL of the compound. The resulting suspension was equilibrated at 25° C. for 24 hours then the pH is measured. The suspension was then filtered through a glass fibre C filter into a 96 well plate. The filtrate was diluted by a factor of 10. Quantification was by HPLC with reference to a standard solution in DMSO. Different volumes of the standard, diluted and undiluted sample solutions were injected. The solubility was calculated using the peak areas determined by integration of the peak found at the same retention time as the principal peak in the standard injection. The results are shown in Table 2, below.

TABLE 2
Solubility of Compounds of Formula I and Formula II.

| Compound | Buffer | Final pH | Solubility (mg/mL) |
|---|---|---|---|
| 103 | Phosphate pH 6.3 | 6.45 | 0.17 |
| | Phosphate pH 6.8 | 7.03 | 0.5200 |
| | Phosphate pH 7.3 | 7.41 | 4.600 |
| | FaSSIF² | 6.61 | 9.100 |
| 101 | FaSSIF | 6.58 | 0.025 |
| Lapatinib | Water | | 0.007 |
| ditosylate¹ | 0.1N HCl | | 0.001 |

¹Solubility of lapatinib ditosylate as reported on FDA approved label.
²FaSSIF stands for “fasted state simulated intestinal fluid” and is a buffer.

Without further description, it is believed that using the preceding description and the illustrative examples, one of ordinary skill in the art can make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 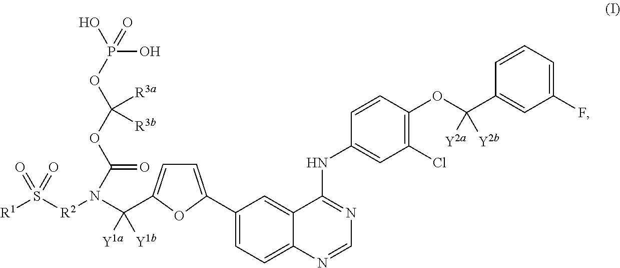
or a pharmaceutically acceptable salt thereof, wherein:
R¹ is selected from —CH₃, —CH₂D, —CHD₂, and —CD₃;
R² is an ethylene moiety, having 0 to 4 deuterium atoms;
each of R^{3a} and R^{3b} is independently selected from hydrogen, C₁-C₆ straight or branched alkyl and C₃-C₇ cycloalkyl, or
R^{3a} and R^{3b} are taken together with the carbon atom to which they are bound to form a C₃-C₇ cycloalkyl, wherein any alkyl or cycloalkyl in R^{3a} or R^{3b} is optionally substituted with halo, C₁₋₇ alkyl, cyano, hydroxyl, carboxy, alkoxy, oxo, amino, alkylamino, or dialkylamino; and
each Y is independently selected from hydrogen and deuterium.

2. The compound of claim 1, wherein any alkyl or cycloalkyl group in R^{3a} or R^{3b} or formed by R^{3a} and R^{3b} taken together with the carbon atom to which they are bound is optionally substituted with halo, C₁₋₇ alkyl, cyano, hydroxyl, carboxy, (C₁₋₇ alkyl)O—, oxo, amino, (C₁₋₇ alkyl)NH—, or (C₁₋₇ alkyl)₂N—.

3. The compound of claim 1, wherein R¹ or R² comprises at least one deuterium; or at least one Y is deuterium.

4. The compound of claim 1, wherein:
R¹ is —CH₃ or —CD₃;
the portion of the compound represented by 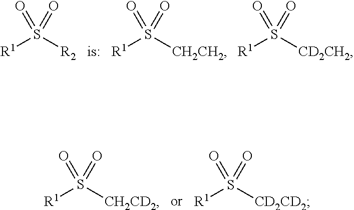
and
R^{3a} and R^{3b} are both hydrogen.

5. The compound of claim 4, wherein:
Y^{1a} and Y^{1b} are both hydrogen; and
Y^{2a} and Y^{2b} are both hydrogen or both deuterium.

6. The compound of claim 4, wherein:
Y^{1a} and Y^{1b} are both deuterium; and
Y^{2a} and Y^{2b} are both hydrogen or both deuterium.

7. The compound of claim 1 which is a compound of Formula II: 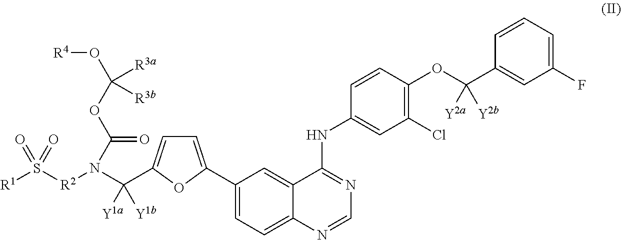
wherein R⁴ is —P(O)₃H₂, —PO₃Na₂, —PO₃K₂, —PO₃(NH₄)₂, —PO₃Ca, or —PO₃Mg.

8. The compound of claim 1, wherein R¹ is CH₃; and R^{3a} and R^{3b} are hydrogen, the compound being selected from any one of the compounds set forth in the table below:

| Compound | R²-†, | Y^{1a}═Y^{1b} | Y^{2a}═Y^{2b} | R⁴ |
|---|---|---|---|---|
| 101 | CH₂CH₂—† | H | H | —PO₃H₂ |
| 102 | CH₂CH₂—† | H | D | —PO₃H₂ |
| 103 | CH₂CH₂—† | H | H | —PO₃Na₂ |
| 104 | CH₂CH₂—† | H | D | —PO₃Na₂ |
| 105 | CH₂CH₂—† | D | H | —PO₃H₂ |
| 106 | CH₂CH₂—† | D | D | —PO₃H₂ |
| 107 | CH₂CH₂—† | D | H | —PO₃Na₂ |
| 108 | CH₂CH₂—† | D | D | —PO₃Na₂ |
| 109 | CD₂CH₂—† | H | H | —PO₃H₂ |
| 110 | CD₂CH₂—† | H | D | —PO₃H₂ |
| 111 | CD₂CH₂—† | H | H | —PO₃Na₂ |
| 112 | CD₂CH₂—† | H | D | —PO₃Na₂ |
| 113 | CD₂CH₂—† | D | H | —PO₃H₂ |
| 114 | CD₂CH₂—† | D | D | —PO₃H₂ |
| 115 | CD₂CH₂—† | D | H | —PO₃Na₂ |
| 116 | CD₂CH₂—† | D | D | —PO₃Na₂ |
| 117 | CH₂CD₂-† | H | H | —PO₃H₂ |
| 118 | CH₂CD₂-† | H | D | —PO₃H₂ |
| 119 | CH₂CD₂-† | H | H | —PO₃Na₂ |
| 120 | CH₂CD₂-† | H | D | —PO₃Na₂ |
| 121 | CH₂CD₂-† | D | H | —PO₃H₂ |
| 122 | CH₂CD₂-† | D | D | —PO₃H₂ |
| 123 | CH₂CD₂-† | D | H | —PO₃Na₂ |
| 124 | CH₂CD₂-† | D | D | —PO₃Na₂ |
| 125 | CD₂CD₂-† | H | H | —PO₃H₂ |
| 126 | CD₂CD₂-† | H | D | —PO₃H₂ |
| 127 | CD₂CD₂-† | H | H | —PO₃Na₂ |
| 128 | CD₂CD₂-† | H | D | —PO₃Na₂ |
| 129 | CD₂CD₂-† | D | H | —PO₃H₂ |
| 130 | CD₂CD₂-† | D | D | —PO₃H₂ |
| 131 | CD₂CD₂-† | D | H | —PO₃Na₂ |
| 132 | CD₂CD₂-† | D | D | —PO₃Na₂ |

wherein “†” indicates point of attachment to the nitrogen atom.

9. The compound of claim 1, selected from:

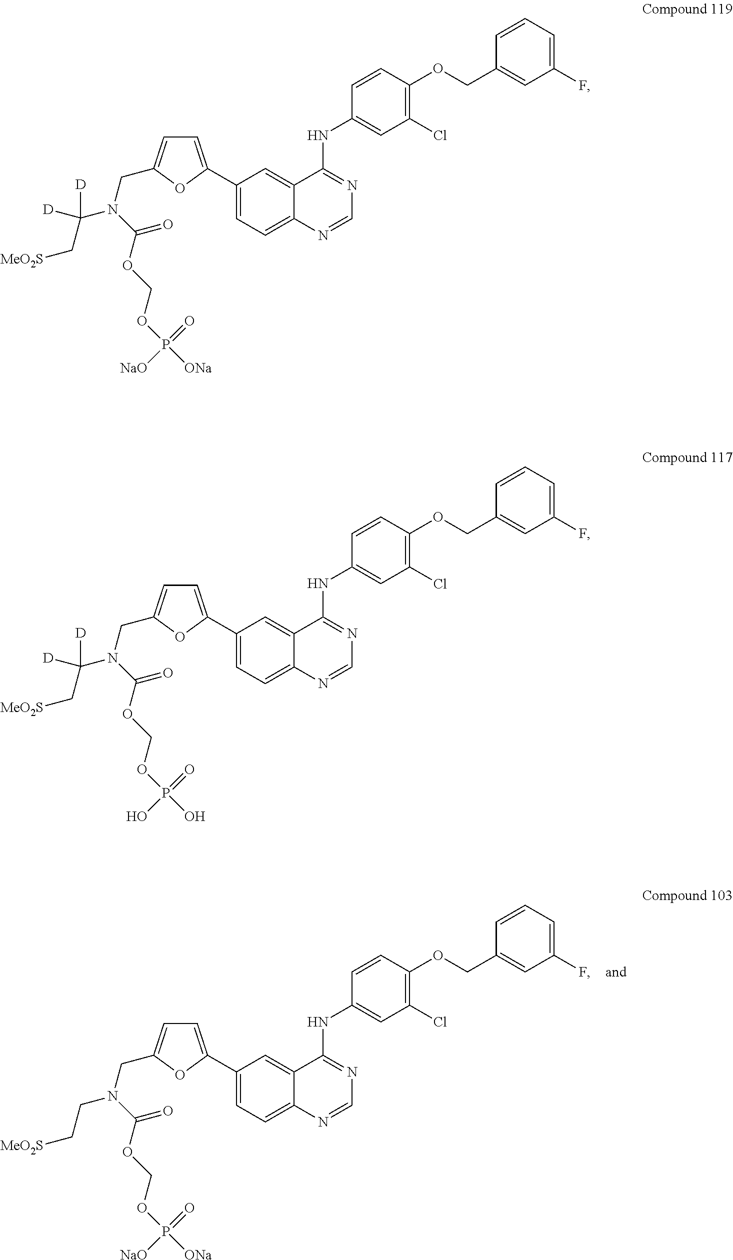

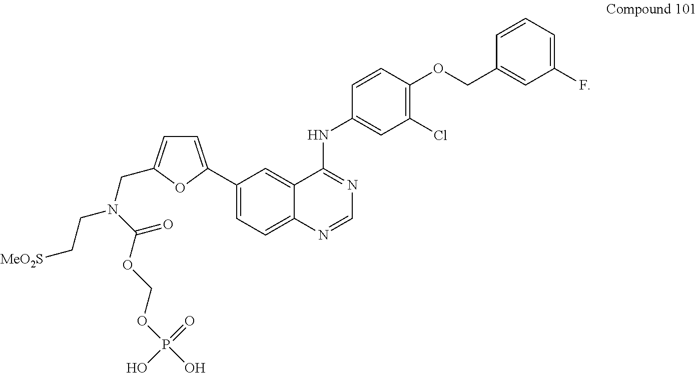

10. The compound of claim 1, wherein any atom not designated as deuterium is present at its natural isotopic abundance.

11. A pyrogen-free pharmaceutical composition comprising a compound of claim 1; and a pharmaceutically acceptable carrier.

12. The composition of claim 11, formulated for oral administration.

13. The composition of claim 12, further comprising an enteric coat surrounding the compound.

14. The composition of claim 11, further comprising a second therapeutic agent selected from an anti-microtubule agents; a platinum coordination complex; an alkylating agent; an antibiotic agents; a topoisomerase II inhibitor; an antimetabolite; a topoisomerase I inhibitor; a hormone or a hormonal analogue; a signal transduction pathway inhibitor; a non-receptor tyrosine kinase angiogenesis inhibitor; an immunotherapeutic agents; a proapoptotic agents; and a cell cycle signaling inhibitor.

15. The composition of claim 14, wherein the second therapeutic agent is selected from capecitabine, pazopanib, trastuzumab, docetaxel, letrozole, tamoxifen, fulvestrant, paclitaxel, carboplatin, bevacizumab, doxorubicin, cyclophosphamide, cisplatin, vinorelbine, everolimus, valproic acid, topotecan, oxaliplatin and gemcitabine.

16. The composition of claim 11 for use in inhibiting the tyrosine kinase activity of ErbB-1 or ErbB-2 in a cell.

17. The composition of claim 11 for use in treating a neoplasia.

18. The composition of claim 17, wherein the neoplasia is selected from acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's, non-Hodgkin's lymphoma, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

19. The composition of claim 18, wherein the neoplasia is selected from breast cancer, esophageal adenocarcinoma, esophageal squamous cell carcinoma, cervical cancer, head and neck cancer, solid tumors, non-Hodgkins' Lymphoma, gastric cancer, ovarian cancer, peritoneal cancer, glioma, glioblastoma multiforme, gliosarcoma, prostate cancer, endometrial cancer, colorectal cancer, non-small cell lung cancer, liver cancer, renal cancer, and pancreatic cancer.

20. The composition of claim 18, wherein the neoplasia is ErbB2-, ErbB4-, or EGF-receptor positive.

21. The composition of claim 20, wherein the neoplasia is ErbB2-, or EGF-receptor positive.

22. The composition of claim 21, wherein the neoplasia is breast cancer.
